(19) United States
(12) Patent Application Publication
(10) Pub. No.: US 2008/0249161 A1
(43) Pub. Date: Oct. 9, 2008

(54) Dermo-Cosmetic Composition For Pets

(75) Inventor: Lionel Fabries, Castres (FR)

Correspondence Address: EGBERT LAW OFFICES, 412 MAIN STREET, 7TH FLOOR, HOUSTON, TX, 77002, US

(73) Assignee: LABORATOIRE DE DERMO-COSMETIQUE ANIMALE (L.D.C.A.) SAS, CASTRES (FR)

(21) Appl. No.: 10/574,760

(22) PCT Filed: Oct. 6, 2004
(86) PCT No.: PCT/FR04/02518

§ 371 Date: Mar. 28, 2008

(30) Foreign Application Priority Data
Oct. 6, 2003 (FR) 0311687

(51) Int. Cl. A61K 31/343

(52) U.S. Cl. 514/450

(57) ABSTRACT

The invention relates to a dermo-cosmetic composition for pets. This composition includes a diffusion solvent vehicle that enables, by a simple application to one or more points of the cutaneous surface, a diffusion over the entirety thereof and at the level of the sebaceous glands. The active ingredients, primarily include a complex of essential oils and poly-unsaturated fatty acids, stored in the sebaceous glands, can be salted out progressively with the sebum product to the surface of the skin.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

This invention concerns a dermo-cosmetic composition for pets (including new “NAC” pets).

### BACKGROUND OF THE INVENTION

Veterinary dermatology products for animal preventive or maintenance usage are generally designed in the form of creams, lotions or shampoos.

Their application to animals is always delicate and sometimes unpleasant for them.

The user's normal trend is often to overdose the quantity of product applied as it is frequently the case for lotions and powders, which causes some disturbance in the animal.

Powders, shampoos or lotions often have a localized effect, which does not make it possible to cover in one single application all needs for a dermo-cosmetic treatment:
skin care and hydration;
soothing effect;
deodorant effect;
sanitizing & purifying effect;
antioxidant and anti-radical effect; and
insect repellent effect.

The purpose of this invention is to eliminate these disadvantages by achieving a composition easier to apply and that in its most elaborate formulation can meet many basic needs of an animal dermo-cosmetic treatment.

The composition under the invention makes it possible after a localized application to diffuse the active ingredients over the whole surface through the diffusion carrier solvent. Then the invention uses the capacity of the sebaceous glands to store in certain conditions the treatment active ingredients and to gradually release them through the natural production of sebum.

To that effect, the dermo-cosmetic composition under the invention, using the capacity of the sebaceous glands to store the active ingredient(s) and to diffuse them through the sebum, is characterized mainly in that it comprises at least:
a carrier that is a solvent, and
a complex of essential oils is selected by themselves or in combination based on the effects to be achieved.

This combination makes it possible to propose a composition ready for use made mainly of a carrier that is a solvent and of essential oils that, once applied in one or more locations of the animal coat, achieves treatment over the whole skin surface of the animal for several days. Tests performed showed that a result was achieved within 7 days minimum.

According to another characteristic of the invention, the solvent making up the carrier is an ethoxydiglycol.

According to another characteristic, the composition comprises:
a carrier consisting of an organic solvent,
a complex of essential oils, and
polyunsaturated fatty acids used locally in animals.

According to a further characteristic of the invention, the above-described composition can contain a soothing agent and/or an anti-inflammatory and/or anti-pruriginous agent.

Other advantages and characteristics of the invention will become obvious when reading the description below.

The purpose of the composition under the invention is to achieve a treatment of animal skin that has several effects:
prevention and hydration;
soothing effect;
deodorant effect;
sanitizing and purifying effect;
antioxidant and anti-radical effect; and
insect-repellent effect.

The advantage of the invention is to be able to combine all or part of the above effects based on the complexity of the composition from an application over a limited area of the animal skin.

### BRIEF SUMMARY OF THE INVENTION

The invention is based on the association of a unique and innovating aromatic synergy with other skin hydrating, soothing, antioxidant and nurturing ingredients. Each essential oil that is part of the composition is subject to a precise qualitative definition based on EOBBD (Essential Oil Botanically and Biochemically Defined, ISO 9002 Certified Quality Assurance Process) criteria.

One single application is sufficient for a small size animal. Two will be necessary for a larger size animal.

According to a basic formulation, the composition under the invention comprises at least one carrier that is made of an organic solvent or equivalent and of a concentrated complex of at least two essential oils.

The presence of the carrier allows for the application of the composition in one or two locations of the skin surface, the diffusion of the product, its storage through the sebaceous glands and its gradual release.

To date, the capacity of the carrier had been used only to provide the animal with an insecticide product. The advantage of the invention is to propose a complete treatment of the skin and coat that has a sanitizing, appeasing and soothing effect on the animal:
Through restoration of a balanced cutaneous ecosystem, and
The invention makes it possible to prevent inflammatory and infectious disorders of the epiderm and the appeasing and soothing effect of the composition provides the animal with a feeling of well-being and freshness that is a mood stabilizer.

The originality of the composition makes it possible to bathe the animal 48 hours before or after the treatment, while still benefitting from the efficiency of the composition applied for one week minimum.

### DETAILED DESCRIPTION OF THE INVENTION

The organic solvent making up the carrier is preferably an intermediate polarity solvent, for example:
an ethoxydiglycol or butylenes glycol, or propylene glycol, or glycerin in a proportion from 1% to 99% of the total composition in relation to its other components.

More generally, the carrier is chosen from the following group:
Acetone, acetonitrile, benzyl alcohol, butyldiglycol, dimethyl acetamide, dimethyl formamide, dipropylene glycol monobutyl ether, ethanol, isopropanol, methanol, ethylene glycol monoethyl ether, monoethyl acetamide, dipropylene glycol monomethyl ether, liquid polyoxyethylene-glycol, propylene glycol, 2-pyrrolidone, including N-methylpyrrolidone, diethyelene glycol monoethyl ether, ethylene glycol, diethyl phtalate, and a mixture of at least two of them.

The above composition can be supplemented by polyunsaturated essential fatty acids to improve the skin surface.

Polyunsaturated fatty acids omega 3 and omega 6 can be introduced by adding hemp seed oil (cannabis sativa) in a proportion that can range from 1% to 15% of the total composition or through natural substitutes such as:
fish oil (orange roughy),
borage oil (Borago Officinalis),
coconut oil (Cocos Nucifera),
kukui oil,
sesame oil (Sesamum indium),
reconstituted synthetic AGPI's or natural extracts,
Eicopentaenoic acid (EPA) and docosahexaenoic acid (DHA),
shea butter,
olive oil (Olea Europea),
canola oil,
walnut oil,
soybean oil, and
anchusa oil (Echium Plantagineum),
   that can be added to the composition in the proportion of 1 to 99%, more specifically 2% to 50% and preferably 5% to 10% of the total composition.

The composition can also contain a soothing and/or anti-inflammatory and/or anti-pruriginous agent.

Preferably, the soothing agent is laureth 9 in the proportion of 0.8% to 3% or its substitutes, such as:
menthol, and its salts, procaine, lidocaine, corticoids, by themselves or in combination of at least two of them.

The laureth 9 polymer is a mixture of polyethylene glycol monolauric ethers with an average of 9 ethylene oxide groups per molecule.

The anti-inflammatory agent can be bisabolol in the proportion of 1% to 5% or its substitutes:
natural or synthetic azulene,
allantoin,
glycyrrhetinic 18 beta acid and its salts in the proportion of 0.01% to 10%, and preferably from 0.01 to 1%, and
aloe extracts,
calendula extracts,
carrot extracts,
   in the proportion of 0.01 to 10%, and preferably from 5 to 10%.

Bisabolol also called alpha-bisabolol has the following formulation:
1-methyl-4(1,5-dimethyl-1 hydroxyhex-4(5)-enyl)-cyclohexen-1;
6-methyl-2-(4-methyl-3-cyclohexen-1-yl)-5-hepten-2 ol.

The above composition contains an antioxidant and anti-radical product: tocopherol or vitamin E, in the form of tocopheryl acetate in the proportion of 0.2% to 2% or its substitutes, such as tocopheryl palmitate or tocopheryl linoleate.

The composition also contains in combination with any one of the above compounds a thickening or stabilizing crystallization-inhibiting film-forming agent, such as polyvinyl pyrrolidone or a product chosen from the following group:
hydroxymethyl cellulose, polyvinyl alcohols, vinyl acetate and vinyl pyrrolidone copolymers, polyethylene glycols, benzyl alcohol, mannitol, glycerol, sorbitol, polyoxyethylene sorbitan esters, lecithin, sodium carboxymethyl cellulose, acrylic derivatives such as methacrylates derived from polyesters of the trimethyl pentanediol/adipic acid type and others.

The composition also contains an insect-repellent product, such as neem seed oil that is in fact Azadirachta indica at 0.25% of azadirachtin or its substitutes such as:
castor oil (Ricinus Communis),
diethyl toluamide and butyl acetylaminopropionate,
dimethyl phtalate,
ethyl hexanediol,
natural and synthetic camphor,
pyrethrum and derivatives (Pyrethum Album), and
oil garlic (Allium Sativum).

More generally, the composition under the invention can contain the following essential oils by themselves or in combination:
Rosmarinus officinalis in the proportion of 0.3% to 0.9% with a purifying and soothing sanitizing effect or eucalyptol.
Lavandula hybrida in the proportion of 0.3% to 0.9% or lavandula angustifolia with a deodorant effect.
Eugenia caryophyllus with an insect-repellent effect, in the proportion of 0.3% to 0.9%.
Melaleuca Alternifolia or origan majorana in the proportion of 0.3% to 0.9% with an anti-infectious effect.
Cinnamorum Camphora or camphor, in the proportion of 0.15% to 0.6% with a deodorant and anti-pruriginous effect.
Mentha Piperita or mentha orvensis in the proportion of 0.15% to 0.6% with a deodorant effect.
Cedrus atlantica or SAS libanus, in the proportion of 0.15% to 0.6% with an anti-parasitic, cicatrizant and anti-carcinogen effect.
Curcuma longa or curcuma zedoaria in the proportion of 0.15% to 0.6% that also has an anti-parasitic effect.
Origanum compactum or origonum vulgare, in the proportion of 0.06% to 0.3% with an insect-repellent and antimicrobial effect.
Gaultheria procumbens or its methyl salicylate substitute with a sedative and anti-pruriginous effect.
Synthetic musk or musk ketone, in the proportion of 0.06% to 0.3% to perfume or mask natural odors.

The above compounds are used in combination in the composition under the invention based on the effects sought.

More generally, the essential oils in the attached list can be combined in the composition under the invention.

ESSENTIAL OIL COMPLEX SUBSTITUTES

| | | | | |
|---|---|---|---|---|
| Ajowan | Almond Tree, | Ammi, Khella | Odorant Dill Seed | Angelica |
| | Bitter | | | |
| White Armose | Basilic | Bay | Bergamot | Rosewood |
| Herb | | | | |
| Sandlewood | Boldo | Bucchu | Cabreuva | Cajeput |
| Calamus | Chamomile, | Camphor | Cinnamon, Ceylon | Cinnamon, China |
| | Roman or | | | |
| | Noble | | | |
| Cardamones | Carrot | Carvi | Catmin | Cedarwood, Atlas |
| Cedarwood, | Celeri | Chenopodium | Rockrose, | Yellow Lemon |
| Virginia | | Vermifuge. | Labdanum-Bearing | |
| Citronnella, | Citronnella, Java | Copahu | Coriander | Cubeb |
| Ceylon | | | | |
| Cumin | Curcuma | Evergreen Cypress | Douglas | Elemi |
| Incense | Black Spruce | Tarragon | Eucalyptus | Lignum Vitae |
| Galbanum | Gaultheria, | Juniper | Geranium, | Ginger |
| | Wintergreen | | Odorant | |
| Clove Tree | Helichrysum | Hyssop with lying | Hyssopus | Inula |
| | | down branches | Officinalis | |

| | | | |
|---|---|---|---|
| Iormenie, Wild | Lantana | Laurus Nobilis | Spike Lavender |
| Chamomile | | | |
| Lavandula Officinalis | Stoechas Lavender | Lavandula Hybrida | Lemongrass |
| Pistacia Lentiscus | Lepstospermum | Sweet Lime | Litsea |
| Lovage | Red Mandarin | Sweet Marjoram | Majorana Sylvestris |
| Wild Marjoram | Matricaria | Larch | Melissa Officinalis |
| Wild Mint | Peppermint | Pennyroyal | Yarrow |
| St. Johns-wort | Nutmeg | Myrrh | Myrtle |
| Spikenard | Niaouli | Orange Tree | Sweet Orange |
| Origanum | Spanish Origanum | Origanum Vulgare | Palmarosa |
| Grapefruit | Patchouli | Parsley | Landes Pine |
| Pinus Syvestris | Black Pepper | Aromatic Vinyl | Rosemarinus Officinalis |
| | | Pyrrolidone | |
| Pyramidal Rosemary | Bulgarian Rose | Sandalwood Amyris | Santolina |
| | | | Dwarf Cypress |
| Balsam Fir | Siberian Fir | Silver Fir | Savory |
| Sassafras | Small Leaf Sage | Salvia Officinalis | Clary |
| Wild Thyme | Blue Tansy | Tea tree | Turpentine |
| Thuya | Thyme | Savory Leaf Thyme | Goldenrod |
| Fleabane | Lemon Verbena | Vetiver | Ylang-ylang, Full |
| | | | Distillation |
| Ylang-ylang, First | | | |
| Batch | | | |

The essential oils were selected based on precise specifications with regard to the active ingredients they contain. Among the active ingredients can be found in particular sesquiterpenes with anti-inflammatory properties, phenol compounds with locally immunostimulating and anti-infectious properties, other phenol compounds (eugenol et carvacrol) with insectifuge and repellent effects and esters with antalgesic and decongestant properties that may temper, even prevent, a possibly irritant action of the phenols on the skin.

A formula especially suited for the composition under the invention contains:
As carrier and skin solvent, an ethoxydiglycol.
As vegetal oil rich in polyunsaturated fatty acids, hemp oil (cannabis sativa).
As concentrated essential oils:
   Rosmarius Officinalis, Lavendula Hybrida,
   Eugenia Caryophyllus, Melaleuca Alternifolia,
   Cinnamomum Camphora, Mentha Piperita,
   Cedrus Atlantica, Curcuma Longa, Origanum Compactum,
   Gaultheria Procumbens, Musk.
As insect repellent and soothing product, neem seed extract (azadirachta Indica) at 0.25% of azadirachtin).
As soothing, desensitizing and anti-pruriginous ingredients, laureth 9.
As thickening, stabilizing crystallization-inhibiting agent forming a film on the animal skin, polyvinyl pyrrolidone as mentioned above in the description.
A vitamin E with antioxidant function, such as D alpha tocopherol or tocopheryl acetate, or the products mentioned above in the description, or the products mentioned above (other antioxidants): butyl hydroxyanisole, butyl hydroxytoluene, ascorbic acid, sodium metabisulfate, propyl gallate, sodium thiosulfate, mixture of no more than two of them.
An anti-inflammatory agent such as alpha-bisabolol, bisabolol or its substitutes as mentioned above in the description.

The formulation active ingredients can be included in whole or in part in a “delay” formulation allowing for extended release time and thus extended action duration of these active ingredients. The compounds allowing for that modification can be chosen among beta-cyclodextrins, silica, cellulose fibers, nylon, poly-methacrylates.

A delay or scheduled release formula can allow for extended release of the active ingredients up to twenty-eight days after application. The delay formulas use:
Beta-cyclodextrins, cyclic natural sugars: present in the formulation with a concentration ranging from 2 to 10%.
And/or cellulose fibers from wood: present in the formulation with a concentration ranging from 2 to 10%.
And/or polymethyl methacrylate and nylon spheres: present in the formulation with a concentration ranging from 4 à 12%.
And/or coated or non-coated silica microreservoirs: present in the formulation with a concentration ranging from 2 to 10%.
And/or linear polyester polymers with a molecular weight of 800 through 5000 Daltons both occlusive and retentive: present in the formulation with a concentration ranging from 10 to 20%.

The proportions of each component are taken in the above-mentioned percentage values, said values being expressed in percentages of the total and full composition.

I claim:

1. Dermo-cosmetic composition using capacity of sebaceous glands to store active ingredients and to release the ingredients through a sebum, the composition comprising:
one carrier being a solvent; and
at least one complex of essential oils.

2. Composition, according to claim 1, wherein said solvent is comprised of an ethoxydiglycol.

3. The composition, according to claim 1, wherein said one carrier is an organic solvent chosen from a group consisting of:
Acetone, acetonitrile, benzyl alcohol, butyldiglycol, dimethyl acetamide, dimethyl formamide, dipropylene glycol monobutyl ether, ethanol, isopropanol, methanol, ethylene glycol monoethyl ether, monoethyl acetamide, dipropylene glycol monomethyl ether, liquid polyoxyethylene-glycol, propylene glycol, 2-pyrrolidone, including N-methylpyrrolidone, diethyelene glycol monoethyl ether, ethylene glycol, diethyl phtalate, and a mixture of at least two organic solvents of the group.

4. The composition, according to claim 1, further comprising:
Polyunsaturated essential fatty acids, wherein said one carrier is an organic solvent.

5. The composition, according to claim 4, wherein the fatty acids are chosen from a group consisting of hemp oils, in a proportion of 1 to 15%.

6. The composition, according to claim 4, wherein the fatty acids are added using products chosen from a group consisting of:
fish oil (orange roughy),
borage oil (Borago Officinalis),
coconut oil (Cocos Nucifera),
kukui oil,
sesame oil (Sesamum indium),
reconstituted synthetic AGPI's or natural extracts,
Eicopentaenoic acid (EPA),
docosahexaenoic acid (DHA),
shea butter,
olive oil (Olea Europea),
canola oil,
walnut oil,
soybean oil, and
anchusa oil (Echium Plantagineum), in a proportion of 3 to 50%, and preferably 5% to 10%.

7. The composition, according to claim 4, further comprising:
a soothing and/or anti-inflammatory and/or anti-pruriginous agent, wherein the fatty acids are omega 3 and omega 6.

8. The composition, according to claim 7, wherein the soothing agent is chosen from a group consisting of:
laureth 9, procaine, lidocaine, corticoids, menthol, and a combination of at least two soothing agents of the group.

9. The composition, according to claim 7, wherein the anti-inflammatory agent is chosen from a group consisting of:
bisabolol, natural or synthetic azulene, allantoin, glycyrrhetinic 18 beta acid and its salts, aloe extracts, calendula extracts, and carrot extracts, in a proportion of 0.01 to 10%.

10. The composition, according to claim 1, further comprising:
a thickening or stabilizing crystallization-inhibiting film-forming agent, the thickening agent being chosen from a group consisting of:
polyvinyl pyrrolidone, hydroxymethyl cellulose, polyvinyl alcohols, vinyl acetate and vinyl pyrrolidone copolymers, polyethylene glycols, benzyl alcohol, mannitol, glycerol, sorbitol, polyoxyethylene sorbitan esters, lecithin, sodium carboxymethyl cellulose, and acrylic derivatives such as methacrylates and others.

11. The composition, according to claim 1, further comprising:
an insect-repellent product being chosen from a group consisting of:
neem seed oil, castor oil (Ricinus Communis), dimethyl phtalate, ethyl hexane diol, natural and synthetic camphor, pyrethrum and derivatives (Pyrethum Album), and oil garlic (Allium Sativum).

12. Composition according to claim 1, wherein said carrier is an ethoxydiglycol, wherein said complex of essential oils is Rosmarius Officinalis, Lavendula Hybrida, Eugenia Caryophyllus, Melaleuca Alternifolia, Cinnamomum Camphora, Mentha Piperita, Cedrus Atlantica, Curcuma Longa, Origanum Compactum, and Gaultheria Procumbens, Musk, said composition further comprising:
hemp oil,
neem seed extract (Azadirachta Indica) at 0.25% of azadirachtin,
laureth 9,
a film-forming means being a thickening, stabilizing crystallization-inhibiting agent,
an antioxidant means, and
an anti-inflammatory means.

13. The composition according to claim 1, further comprising:
a delay formula allowing for extended release of the active ingredients.

14. Composition according to claim 13, wherein said delay formula is chosen from a group consisting of:
Beta-cyclodextrins, as cyclic natural sugars having a concentration ranging from 2 to 10%, cellulose fibers from wood having a concentration ranging from 2 to 10%, polymethyl methacrylate and nylon spheres having a concentration ranging from 4 to 12%, coated or non-coated silica microreservoirs having a concentration ranging from 2 to 10%, linear polyester polymers with a molecular weight of 800 through 5000 Daltons both occlusive and retentive having a concentration ranging from 10 to 20%, and a combination of at least two of the delay formula of the group.
